(19) United States
(12) Patent Application Publication
(10) Pub. No.: US 2008/0171655 A1
(43) Pub. Date: Jul. 17, 2008

(54) PROCESS FOR PREPARING A CATALYST

(76) Inventors: Edward Julius Creyghton, Amsterdam (NL); Carolus Matthias Anna Maria Mesters, Amsterdam (NL); Marinus Johannes Reynhout, Amsterdam (NL); Guy Lode Magda Maria Verbist, Amsterdam (NL)

Correspondence Address: Shell Oil Company, 910 Louisiana, Houston, TX, 77002, US

(21) Appl. No.: 11/954,346

(22) Filed: Dec. 12, 2007

(30) Foreign Application Priority Data
Dec. 12, 2006 (EP) 06125910.7

(51) Int. Cl. C07C 1/02; B01J 23/75; B01J 23/34

(52) U.S. Cl. 502/325; 502/324; 518/715; 502/337; 502/338

(57) ABSTRACT

A process for the preparation of a catalyst precursor for use in a reactor, comprising the steps of:
(a) coating one or more promoter(s) and/or one or more co-catalyst(s) onto a carrier material to form a coated carrier material;
(b) combining a catalyst material with the coated carrier material of step (a); and
(c) calcining the material of step (b).

The present invention relates to a process for preparing a catalyst precursor and catalyst for use in producing normally gaseous, normally liquid and optionally solid hydrocarbons from synthesis gas, generally provided from a hydrocarbonaceous feed, for example a Fischer-Tropsch process.

Many documents are known describing processes for the catalytic conversion of (gaseous) hydrocarbonaceous feedstocks, especially methane, natural gas and/or associated gas, into liquid products, especially methanol and liquid hydrocarbons, particularly paraffinic hydrocarbons.

The Fischer-Tropsch process can be used as part of the conversion of hydrocarbonaceous feed stocks into liquid and/or solid hydrocarbons. Generally the feed stock (e.g. natural gas, associated gas, coal-bed methane, coal, heavy and/or residual oil fractions, biomass, etc.) is converted in a first step into a mixture of hydrogen and carbon monoxide (often referred to as synthesis gas or syngas). The synthesis gas is then fed into a reactor where it is converted in one or more steps over a suitable catalyst at elevated temperature and pressure into compounds ranging from methane to high molecular weight modules comprising up to 200 carbon atoms, or, under particular circumstances, even more, (and water).

Catalysts used in the Fischer-Tropsch synthesis often comprise a carrier based support material and one or more metals from Group VIII of the Periodic Table, especially from the cobalt and iron groups, optionally in combination with one or more metal oxides and/or metals as promoters selected from zirconium, titanium, chromium, vanadium and manganese, especially manganese. Such catalysts are known in the art and have been described for example, in the specifications of WO 9700231A and U.S. Pat. No. 4,595,703.

General methods of preparing catalyst materials and forming catalyst mixtures are known in the art, see for example U.S. Pat. No. 4,409,131, U.S. Pat. No. 5,783,607, U.S. Pat. No. 5,502,019, WO 0176734, CA 1166655, U.S. Pat. No. 5,863,856 and U.S. Pat. No. 5,783,604. These include preparation by co-precipitation and impregnation. Such processes could also include freezing, sudden temperature changing, etc.

Catalysts for the Fischer-Tropsch process are usually prepared by obtaining a metal hydroxide, very carefully oxidising it to the metal oxide and then placing it in the appropriate reactor where it is reduced to the metal in situ.

One catalytically active metal for Fischer-Tropsch reactions is cobalt, and one common promoter is manganese. One traditional example of their combination is the mixing of titania with cobalt oxide, shaping the mixture and then impregnating it with manganese acetate. However, impregnation is not only restricted by the pore volume of the carrier, but in practice, several impregnation steps are needed to obtain the desired loading of manganese, and the need for such a number of steps is undesirable in the preparation of the catalysts on a commercial scale. Moreover, there is no good control of where the manganese is sitting in the carrier, i.e. its distribution and density. Thus, such catalysts have sometimes been termed ‘egg shell’ catalysts, as there may be no penetration of the promoter into the carrier beyond its outer ‘shell’.

In another known preparation of a cobalt and manganese catalyst, manganese is carefully precipitated with cobalt hydroxide to form a solid solution of (Co,Mn)(OH)₂, which can be used as a starting material. This material is mixed with titania and extruded. It is then calcined and decomposed to form the cobalt manganese oxide (Co,Mn)O), and then further oxidised to (Co,Mn)₃O₄). In a Fischer-Tropsch reactor, the cobalt oxide is reduced to its base metal form.

However, the forming, calcining and subsequent reduction of the cobalt manganese solid solution are delicate processes. Control of solid solutions is difficult in its own right. Because of this, it is difficult to properly achieve the desired final cobalt manganese balance.

It is one object of the present invention to provide an improved catalyst and catalyst precursor, and an easier process for making them.

According to one aspect of the present invention, there is provided a process for the preparation of a catalyst precursor for use in a reactor, comprising the steps of:

(a) coating one or more promoter(s) and/or one or more co-catalyst(s) onto a carrier material to form a coated carrier material;
(b) combining a catalyst material with the coated carrier material of step (a); and
(c) calcining the material of step (b).

The catalyst material is generally based on a catalytically active metal such as cobalt, iron, nickel and ruthenium, preferably cobalt.

The catalyst material may also include one or more promoters and/or one or more co-catalysts therewith prior to its combination with the coated carrier material. The amount of promoter(s) and/or co-catalyst(s) may be a proportion or fraction of the total amount of promoter(s) and/or co-catalyst(s) desired in the final catalyst precursor or catalyst itself.

Thus, the present invention extends from

(a) a process wherein all the desired promoter(s) and/or co-catalyst(s) in the catalyst precursor is made available to the catalytically active metal from the coated carrier material, to
(b) a process wherein a part of the promoter(s) and/or co-catalyst(s) in the catalyst precursor is available to the catalytically active metal from the carrier material, and a part is available as part of the starting catalyst material.

The introduction of promoter(s) and/or co-catalyst(s) onto the carrier material provides a separate route for the introduction, generally by migration, of the promoter(s) and/or co-catalyst(s) into the catalyst material.

The present invention can therefore reduce, and optionally eliminate, the need for substances such as the promoter(s) and/or co-catalyst(s) to be provided completely separately, for example through impregnation, or as co-precipitates, especially for combined metal compound materials that are difficult to reduce or decompose to form the desired and correct oxide material, and then bare metal material.

In contrast, the decomposition (during calcination) of single metal compounds such as cobalt hydroxide is a well known process, which is easily controllable from easily obtainable starting materials.

Thus, the present invention provides a process for the preparation of a catalyst precursor using easier starting materials and easier process steps.

Another advantage of the present invention is flexibility for the introduction of promoters and co-catalysts with a catalyst material. By introducing the promoter(s) and/or co-catalyst(s) onto the carrier material, which process is a simple process that can be easily carried out, any complex interaction of the promoter(s) and co-catalyst(s) with the catalyst material is avoided, as the catalyst material is added (either wholly or at least partly) as a separate substance.

Where the catalyst material still includes a proportion or fraction of one or more promoters and/or co-catalysts (as well as the promoter(s) and/or co-catalyst(s) coated on the carrier material), the present invention also provides increased flexibility as to the proportions and ratios of the promotor(s) and/or co-catalyst(s) on the carrier material or with the catalyst material, increasing the flexibility of how to introduce the substances together to form the catalyst precursor. Such flexibility is not possible when using conventional solid solutions.

A further advantage of the present invention is the simplicity of combining a catalyst material with a coated carrier material. A number of simple processes are useable for this combining, including for example co-milling or co-mulling. This can increase the ease of the combining, such as possible amounts or homogeneity of the components. This can be directly contrasted with combining and calcining solid Co,Mn oxide solutions with titania, and their difficult reduction to base metal forms.

The simplicity of combining the catalyst material with the carrier material is especially advantageous where some processes such as impregnation can be limited. For example, a common carrier material is titania, which is known to have a low porosity compared with for example silica, another common carrier material. Thus, it is common that multiple impregnations of catalyst material are required to achieve a desired or pre-determined level of catalyst material in the titania carrier material. The present invention can use simpler combining processes for the catalyst material and the carrier material to avoid this problem.

Suitable promoters for use with the present invention can be one or more metals and/or metal oxides. Suitable metal oxides may be selected from Groups IIA, IIIB, IVB, VB, VIB, VIIB and VIIIB of the Periodic Table of Elements, or the actinides and lanthanides. In particular, oxides of tungsten, magnesium, calcium, strontium, barium, scandium, yttrium, lanthanum, cerium, titanium, zirconium, hafnium, thorium, uranium, vanadium, chromium and manganese are most suitable promoters.

References to “Groups” and the Periodic Table as used herein relate to the previous IUPAC version of the Periodic Table of Elements such as that described in the 68^{th} Edition of the Handbook of Chemistry and Physics (CPC Press).

Suitable promoters which are metals may be selected from Groups VIIB or VIII of the (same) Periodic Table. Manganese, iron, tungsten, rhenium and Group VIII noble metals are particularly suitable, with platinum and palladium being especially preferred.

Combinations of two or more promoters being metals or metal oxides may also be used.

Suitable co-catalysts include one or more metals such as iron, nickel, or one or more noble metals from Group VIII. Preferred noble metals are platinum, palladium, rhodium, ruthenium, iridium and osmium. Most preferred co-catalysts for use in hydro-cracking are those comprising platinum. Such co-catalysts are usually present in small amounts.

A most suitable catalyst comprises cobalt as the catalytically active metal and zirconium as a promoter. Another most suitable catalyst comprises cobalt as the catalytically active metal and manganese and/or vanadium as promoter(s).

The promoter(s) and/or co-catalyst(s) can be provided in any suitable form. Generally, metal promoters are provided in a compound form, more usually as a salt. Examples include acetates, nitrates, halides, etc. The provision of, for example, manganese acetate is a well-known compound.

The coating of the promoter(s) and/or co-catalyst(s) onto the carrier material can be carried out by any process known in the art, such as locating the carrier material with a solution of the promoter(s) compound and/or co-catalyst(s), and then allowing the coated catalyst material to dry.

It is preferred to apply a mono-layer (i.e. single atomic layer) of promoter(s) and/or co-catalyst(s) onto the carrier material.

The coating may not cover the carrier material completely, either as desired or as a result of the coating step.

Any promoter(s) is typically present in an amount of from 0.1 to 60 parts by weight per 100 parts by weight of any carrier material used. It will however be appreciated that the optimum amount of promoter(s) may vary for the respective elements which act as promoter(s). If the intended catalyst comprises cobalt as the catalytically active metal and manganese and/or vanadium as promoter(s), the cobalt: (manganese+vanadium) atomic ratio is advantageously at least between 5:1 and 30:1.

In one embodiment of the present invention, the catalyst comprises the promoter(s) and/or co-catalyst(s) having a concentration in the Group VIII metal(s) in the range 0.1-10 atom % (based on cobalt), preferably 0.3-7 atom %, and more preferably 0.4-6 atom %.

In particular, the present invention can provide a cobalt catalyst, especially a manganese or vanadium promoted cobalt catalyst, formed by dispersing or co-milling the cobalt or cobalt compound, or cobalt and manganese or vanadium compounds, upon a titanic, alumina, or quartzite support which has been pre-coated with manganese and/or vanadium.

The ratio of promoter(s) and/or co-catalyst(s) available to the catalytically active metal from the coated carrier material can range from a de minimus proportion, such as for example (but not limited to) 0.01 atom %, to 100 atom %.

Preferably, the concentration of the promoter(s) and/or co-catalyst(s) on the catalyst material is less than 1 atom % (based on cobalt), optionally in combination with use of a catalyst material that is absent any promoter(s) and/or co-catalyst(s) prior to step (b).

It is known in the art that the amount or concentration of promoter(s) and/or co-catalyst(s) relates to the desired use or effect of the catalyst material, which is usually related to the operating conditions of the reactor, and possibly the nature of the starting material in the reactor.

Preferably in the present invention, the catalytically active metal for the catalyst material is provided in the form of a hydroxide, carbonate, citrate, nitrate, oxyhydroxide or oxide, more preferably hydroxide.

As one example, cobalt hydroxide is a common material which is readily available, and its decomposition to, for example, cobalt oxide is a relatively easy process. Hitherto, the combination of a promoter such as manganese with cobalt had to be created in the form of a co-precipitate and solid solution. Creating co-precipitates of some metals is not a simple process, and can easily be mishandled. Moreover, the decomposition of solid solutions is again a complex process which requires careful handling and again can easily create undesired final products. On an industrial scale, the availability and easy decomposition of compounds such as cobalt hydroxide provide a significantly easier, and therefore more economic, process in catalyst manufacture.

During the calcination step (c), the promotor(s) and/or co-catalyst(s) coated on the carrier material migrates or diffuses into the catalyst material so as to be in combination with the catalytically active metal as hitherto provided. The catalytically active metal and promotor(s) and/or co-catalyst(s) can then operate or co-operate in the same manner on the material or materials in the reactor in the same manner as prior catalysts.

The carrier material may be selected from any of the suitable refractory metal oxides or silicates or combinations thereof known in the art. Particular examples of preferred porous carriers include silica, alumina, titania, zirconia, ceria, gallia and mixtures thereof, especially silica and titania.

The optimum amount of catalytically active metal with the carrier material depends inter alia on the specific catalytically active metal. Typically, the amount of for example cobalt present in the catalyst may range from 1 to 100 parts by weight per 100 parts by weight of carrier material, preferably from 10 to 50 parts by weight per 100 parts by weight of carrier material.

Titania is a suitable carrier material. With titania, the catalyst material may further comprise up to 20% by weight of another refractory oxide, typically silica, alumina or zirconia, or a clay as a binder material, preferably up to 10% by weight based on the total weight of refractory oxide and binder material. Preferably, the titania has been prepared in the absence of sulphur-containing compounds. An example of such preparation method involves flame hydrolysis of titanium tetra-chloride. Titania is available commercially and is well-known as material for use in the preparation of catalysts or catalyst precursors. The titania suitably has a surface area of from 0.5 to 200 m²/g, more preferably of from 20 to 150 m²/g.

If desired, the catalyst material such as a cobalt compound may still be combined with a promoter(s), (whose proportion is therefore reduced in a complementary relationship with the proportion of promoter(s) on the carrier material). Compounds of for example cobalt and promoter metal can be obtained by co-precipitation, usually co-precipitation at constant pH. Co-precipitation at constant pH may be performed by the controlled addition of a base, a base-releasing compound, an acid or an acid-releasing compound to a solution comprising a soluble cobalt compound and a soluble compound of promoter metal, preferably by the controlled addition of ammonia to an acidic solution of a cobalt compound and a promoter metal compound.

With the amount of any promoter(s) in the co-precipitation process reduced (for example to <5, 4, 3, 2, 1 or even <1 wt %), compared with the amount used in co-precipitates conventionally prepared and used (5 or 6 wt %), the co-precipitation process is naturally easier to carry out, and easier to reduce to base metal form. Such processes are therefore advantageously more successful on an industrial scale.

Step (b) of the process of the present invention may suitably be carried out by methods known to those skilled in the art, such as by kneading, mulling or stirring.

Typically, the ingredients of step (b) are mulled for a period of from 5 to 120 minutes, preferably from 15 to 90 minutes. During the mulling process, energy is put into the mixture by the mulling apparatus. The mulling process may be carried out over a broad range of temperature, preferably from 15 to 90° C. As a result of the energy input into the mixture during the mulling process, there will be a rise in temperature of the mixture during mulling. The mulling process is conveniently carried out at ambient pressure. Any suitable, commercially available mulling machine may be employed.

To improve the flow properties of the mixture of step (b), it is preferred to include one or more flow improving agents and/or extrusion aids in the mixture prior to extrusion. Suitable additives for inclusion in the mixture include fatty amines, quaternary ammonium compounds, polyvinyl pyridine, sulphoxonium, sulphonium, phosphonium and iodonium compounds, alkylated aromatic compounds, acyclic mono-carboxylic acids, fatty acids, sulphonated aromatic compounds, alcohol sulphates, ether alcohol sulphates, sulphated fats and oils, phosphonic acid salts, polyoxyethylene alkylphenols, polyoxyethylene alcohols, polyoxyethylene alkylamines, polyoxyethylene alkylamides, polyacrylamides, polyols and acetylenic glycols. Preferred additives are sold under the trademarks Nalco and Superfloc.

To obtain strong extrudates, it is preferred to include in the mixture, prior to any shaping step, for example by extrusion, at least one compound which acts as a peptising agent for the titania. Suitable peptising agents for inclusion in the extrudable mixture are well known in the art and include basic and acidic compounds. Examples of basic compounds are ammonia, ammonia-releasing compounds, ammonium compounds or organic amines. Such basic compounds are removed upon calcination and are not retained in the extrudates to impair the catalytic performance of the final product. Preferred basic compounds are organic amines or ammonium compounds. A most suitable organic amine is ethanol amine. Suitable acidic peptising agents include weak acids, for example formic acid, acetic acid, citric acid, oxalic acid, and propionic acid.

Optionally, burn-out materials may be included in the mixture, prior to extrusion, in order to create macropores in the resulting extrudates. Suitable burn-out materials are commonly known in the art.

The total amount of flow-improving agents/extrusion aids, peptising agents, and burn-out materials in the mixture preferably is in the range of from 0.1 to 20% by weight, more preferably from 0.5 to 10% by weight, on the basis of the total weight of the mixture.

Extrusion may be effected using any conventional, commercially available extruder. In particular, a screw-type extruding machine may be used to force the mixture through the orifices in a suitable dieplate to yield extrudates of the desired form. The strands formed upon extrusion may be cut to the desired length.

After any shaping step, the shaped products, for example extrudates, may be dried. Drying may be effected at an elevated temperature, preferably up to 500° C., more preferably up to 300° C. The period for drying is typically up to 5 hours, more preferably from 15 minutes to 3 hours.

In another embodiment of the invention, the solids contents of the mixture obtained in step (b) is such that a slurry or suspension is obtained, and the slurry or suspension thus-obtained is shaped and dried by spray-drying. The solids content of the slurry/suspension is typically in the range of from 1 to 30% by weight, preferably of from 5 to 20% by weight.

The extruded and dried, spray-dried or otherwise-shaped and dried compositions are subsequently calcined in step (c). Calcination is effected at elevated temperature, preferably at a temperature between 350 and 750° C., more preferably between 450 and 650° C. The duration of the calcination treatment is typically from 5 minutes to several hours, preferably from 15 minutes to 4 hours. Suitably, the calcination treatment is carried out in an oxygen-containing atmosphere, preferably air. It will be appreciated that, optionally, the drying step and the calcining step can be combined.

The resulting catalyst precursor may be activated by contacting the catalyst with hydrogen or a hydrogen-containing gas, typically at temperatures of about 200 to 450° C.

The present invention extends to the activation of a catalyst precursor prepared as herein described, particularly but not exclusively, by decomposition of the catalyst material and/or reduction of the catalyst material to its metal form. The invention also extends to a catalyst formed thereby.

A catalyst provided by the present invention is particularly, but not exclusively, useful for a hydrocarbon synthesis process such as a Fischer-Tropsch reaction. Fischer-Tropsch catalysts are known in the art, and as a Group VIII metal component, they preferably use cobalt, iron and/or ruthenium, more preferably cobalt.

A steady state catalytic hydrocarbon synthesis process may be performed under conventional synthesis conditions known in the art. Typically, the catalytic conversion may be effected at a temperature in the range of from 100 to 600° C., preferably from 150 to 350° C., more preferably from 180 to 270° C. Typical total pressures for the catalytic conversion process are in the range of from 1 to 200 bar absolute, more preferably from 10 to 70 bar absolute. In the catalytic conversion process mainly C₅+ hydrocarbons are formed, based on the total weight of hydrocarbonaceous products formed, (at least 70 wt %, preferably 90 wt %).

According to a second aspect of the present invention, there is provided a process for producing normally gaseous, normally liquid and optionally normally solid hydrocarbons from synthesis gas which comprises the steps of:

(i) providing the synthesis gas; and
(ii) catalytically converting the synthesis gas of step (i) at an elevated temperature and pressure to obtain the normally gaseous, normally liquid and optionally normally solid hydrocarbons;
wherein the catalyst for step (ii) is formed from a performer herein described.

The present invention also provides a process further comprising:

(iii) catalytically hydrocracking higher boiling range paraffinic hydrocarbons produced in step(ii),
as well as hydrocarbons whenever provided by a process as described herein.

The present invention also provides use of a catalyst as defined herein in a process for producing normally gaseous, normally liquid and optionally normally solid hydrocarbons from synthesis gas which comprises the steps of:

(i) providing the synthesis gas; and
(ii) catalytically converting the synthesis gas of step (i) at an elevated temperature and pressure to obtain the normally gaseous, normally liquid and optionally normally solid hydrocarbons.

Fischer-Tropsch synthesis is preferably carried out at a temperature in the range from 125 to 350° C., more preferably 175 to 275° C., most preferably 180° C. to 260° C. The pressure preferably ranges from 5 to 150 bar abs., more preferably from 5 to 80 bar abs.

Preferably, a Fischer-Tropsch catalyst is used, which yields substantial quantities of paraffins, more preferably substantially unbranched paraffins. A part may boil above the boiling point range of the so-called middle distillates, to normally solid hydrocarbons. A most suitable catalyst for this purpose is a cobalt-containing Fischer-Tropsch catalyst. The term “middle distillates”, as used herein, is a reference to hydrocarbon mixtures of which the boiling point range corresponds substantially to that of kerosene and gas oil fractions obtained in a conventional atmospheric distillation of crude mineral oil. The boiling point range of middle distillates generally lies within the range of about 150 to about 360° C.

The higher boiling range paraffinic hydrocarbons if present, may be isolated and subjected to a catalytic hydrocracking step, which is known per se in the art, to yield the desired middle distillates. The catalytic hydrocracking is carried out by contacting the paraffinic hydrocarbons at elevated temperature and pressure and in the presence of hydrogen with a catalyst containing one or more metals having hydrogenation activity, and supported on a carrier. Suitable hydrocracking catalysts include catalysts comprising metals selected from Groups VIB and VIII of the (same) Periodic Table of Elements. Preferably, the hydrocracking catalysts contain one or more noble metals from Group VIII. Preferred noble metals are platinum, palladium, rhodium, ruthenium, iridium, and osmium. Most preferred catalysts for use in the hydrocracking stage are those comprising platinum.

The amount of catalytically active metal present in the hydrocracking catalyst may vary within wide limits and is typically in the range of from about 0.05 to about 5 parts by weight per 100 parts by weight of the carrier material. Suitable conditions for the catalytic hydrocracking are known in the art. Typically, the hydrocracking is effected at a temperature in the range of from about 175 to 400° C. Typical hydrogen partial pressures applied in the hydrocracking process are in the range of from 10 to 250 bar.

The process may be operated in a single pass mode (“once through”) or in a recycle mode. Slurry bed reactors, ebulliating bed reactors and fixed bed reactors may be used, the fixed bed reactor being the preferred option.

The product of the hydrocarbon synthesis and consequent hydrocracking suitably comprises mainly normally liquid hydrocarbons, beside water and normally gaseous hydrocarbons. By selecting the catalyst and the process conditions in such a way that especially normally liquid hydrocarbons are obtained, the product obtained (“syncrude”) may transported in the liquid form or be mixed with any stream of crude oil without creating any problems as to solidification and or crystallization of the mixture. It is observed in this respect that the production of heavy hydrocarbons, comprising large amounts of solid wax, are less suitable for mixing with crude oil while transport in the liquid form has to be done at elevated temperatures, which is less desired.

The off gas of the hydrocarbon synthesis may comprise normally gaseous hydrocarbons produced in the synthesis process, nitrogen, unconverted methane and other feedstock hydrocarbons, unconverted carbon monoxide, carbon dioxide, hydrogen and water. The normally gaseous hydrocarbons are suitably C₁₋₅ hydrocarbons, preferably C₁₋₄ hydrocarbons, more preferably C₁₋₃ hydrocarbons. These hydrocarbons, or mixtures thereof, are gaseous at temperatures of 5-30° C. (1 bar), especially at 20° C. (1 bar). Further, oxygenated compounds, e.g. methanol, dimethyl ether, may be present in the off gas. The off gas may be utilized for the production of electrical power, in an expanding/combustion process such as in a gas turbine described herein, or recycled to the process. The energy generated in the process may be used for own use or for export to local customers. Part of an energy could be used for the compression of the oxygen containing gas.

The process as just described may be combined with all possible embodiments as described in this specification.

Any percentage mentioned in this description is calculated on total weight or volume of the composition, unless indicated differently. When not mentioned, percentages are considered to be weight percentages. Pressures are indicated in bar absolute, unless indicated differently.

The invention will now be illustrated further by means of the following Examples of catalyst precursors.

### EXAMPLES

### Comparative Example

A mixture was prepared containing 2200 g commercially available titania powder (P25 ex. Degussa), 1000 g of prepared CoMn(OH)ₓ co-precipitate (atomic ratio of Mn/Co is 0.05), 900 g of a 5 wt % polyvinyl alcohol solution and a solution consisting of 300 g water and 22 g of an acidic peptizing agent. The mixture was kneaded for 18 minutes. The loss on ignition (LOI) of the mix was 33.0 wt %. The mixture was shaped using a 1-inch Bonnot extruder, supplied with a 1.7 mm trilob plug. The extrudates were dried for 16 hours at 120° C. and calcined for 2 hours at various temperatures.

### Example 1

1200 g of commercially available titania powder (P25 ex. Degussa) was coated with a solution of 52 g of manganese acetate tetrahydrate in water so as to produce a 1 wt % manganese-coated titania powder. 112.63 g of this powder was mulled for 160 minutes with 50.14 g of cobalt hydroxide, 3.0 g PVA, 1.5 g of citric acid and 67 g of water. The mulled mixture was shaped using a Bonnet extruder, and the extrudates dried for two hours at 120° C., followed by calcination for two hours at 550° C. The resulting extrudates contained 20 wt % cobalt, and 0.68 wt % of manganese.

### Example 2

600 g of commercially available titania powder (P25 ex. Degussa) was coated with a solution of 13.5 g of ammonium metavanadate in water so as to produce a 1 wt % vanadium-coated titania powder. 112 g of this powder was mulled for 60 minutes with 50.10 g of cobalt hydroxide, 3.0 g PVA, 1.5 g of citric acid and 60 g of water. The mulled mixture was shaped using a Bonnet extruder, and the extrudates dried for two hours at 120° C., followed by calcination for two hours at 550° C. The resulting extrudates contained 20 wt % cobalt, and 0.71 wt % of vanadium.

### Example 3

400 g of commercially available titania powder (P25 ex. Degussa) was coated with a solution of 4.5 g of ammonium metavanadate and 8.7 g of manganese acetate tetrahydrate in water so as to produce a 0.5 wt % vanadium-coated and 0.5 wt % manganese titania powder. 112 g of this powder was mulled for 60 minutes with 50.10 g of cobalt hydroxide, 3.0 g PVA, 1.5 g of citric acid and 60 g of water. The mulled mixture was shaped using a Bonnot extruder, and the extrudates dried for two hours at 120° C., followed by calcination for two hours at 550° C. The resulting extrudates contained 20 wt % cobalt, 0.38 wt % of vanadium and 0.35 wt % manganese.

Thus, Comparative Example catalyst material was based on a co-precipitate of cobalt and manganese of 6 atom %, whose hydroxide form was decomposed to the oxide, and then to the base metal. Meanwhile, Examples 1-3 all have about a 79% titania, about 20% cobalt, with the remainder being primarily one or more of the promoters.

The catalyst precursors of the Comparative Example and of Examples 1, 2 and 3 were placed in separate Fischer-Tropsch reactors, activated, and then used with the same process and flow conditions (100 run hours in a Fischer-Tropsch reactor, with GHSV 1200, average temperature 214° C., and a standard H₂/CO ratio.)

The catalysts based on Examples 1, 2 and 3 were found to have similar, if not better, STY activity over several hundred running hours of the reactor as shown in Table 1.

TABLE 1

| | | | | Total |
|---|---|---|---|---|
| | | | | Promoter |
| | STY | C₅₊ | | (for |
| | g/(CH₂)n/NI/hr | selectivity | CO₂ % | 20 wt % Co) |
| Comparative | 154 | 92 | 1.8% | 1% Mn |
| Example | | | | |
| Co + Mn | | | | |
| Example 1 | 149 | 92.8 | 1.0% | 0.68% Mn |
| Mn Coating | | | | on TiO₂ |
| Example 2 | 162 | 92.6 | 1.6% | 0.71% V on |
| V Coating | | | | TiO₂ |
| Example 3 | 173 | 92 | 1.4% | 0.35% Mn |
| Mn + V | | | | 0.38% V |
| Coating | | | | on TiO₂ |

Thus, by introducing the promoter or co-promoters in the final catalyst via the coating on the carrier material (being in these Examples titania), the activity of catalyst so formed has not been affected, and indeed for Examples 2 and 3, it has been improved.

A comparison study was also made of the percentage weight of carbon dioxide formed by the catalysts in Table 1 above under the same Fischer-Tropsch reaction conditions. Carbon dioxide is an undesired by-product of the Fischer-Tropsch reaction, and takes away carbon from the production of hydrocarbons.

Table 1 shows that the Comparative Example prior art material produced a CO₂ percentage of about 1.8 over a running time of at least several hundred hours. By comparison, the catalyst materials of the present invention in Table 1 reduced this percentage to about 1 or less than 1, over the same time period.

Thus, each catalyst of Examples 1, 2 and 3 reduced the carbon dioxide formed compared with the conventional Comparative Example, Example 1 particularly. This is a significant reduction on a industrial scale.

1. A process for the preparation of a catalyst precursor for use in a reactor, comprising the steps of:
(a) coating one or more promoter(s) and/or one or more co-catalyst(s) onto a carrier material to form a coated carrier material;
(b) combining a catalyst material with the coated carrier material of step (a); and
(c) calcining the material of step (b).

2. A process as claimed in claim 1 wherein the combined material of step (b) is shaped prior to step (c).

3. A process as claimed in claim 1 or claim 2 wherein the catalyst material is based on a catalytically active metal being one or more selected from the group comprising: cobalt, iron, nickel and ruthenium;
preferably cobalt.

4. A process as claimed in one or more of the preceding claims wherein the promoter(s) and/or co-catalyst(s) are one or more selected from the group comprising: titanium, tungsten, zirconium, manganese, vanadium, rhenium, or an oxide thereof, or a combination of one or more of said metals or their oxides; preferably manganese and/or vanadium.

5. A process as claimed in any one of the preceding claims wherein the catalyst material includes one or more promoters and/or one or more co-catalysts.

6. A process as claimed in any one of the preceding claims wherein the carrier material is a refractory metal oxide, including titania, aluminia, silica, and mixtures thereof, preferably titania.

7. A process as claimed in any one of the preceding claims wherein the concentration of promoter(s) and/or co-catalyst(s) on the carrier material is in the range 0.1-20% atom of cobalt, preferably 0.3-7% atom, and more preferably 0.4-6% atom.

8. A process as claimed in claim 7 wherein the concentration of the promoter(s) and/or co-catalyst(s) on the carrier material is less than 1 of the total weight of the catalyst precursor.

9. A process as claimed in any one of the preceding claims the promoter(s) and/or co-catalyst(s) forms a mono-layer on the carrier material.

10. A process for preparing a catalyst wherein the catalyst precursor as defined in any one of the preceding claims is activated by reduction.

11. A process for producing normally gaseous, normally liquid and optionally normally solid hydrocarbons from synthesis gas which comprises the steps of:
(i) providing the synthesis gas; and
(ii) catalytically converting the synthesis gas of step (i) at an elevated temperature and pressure to obtain the normally gaseous, normally liquid and optionally normally solid hydrocarbons;
wherein the catalyst for step (ii) is formed from a catalyst precursor as defined in any one of the claim 1 to 9, or is a catalyst as defined in claim 10.

12. A process as claimed in claim 11 wherein the process further comprises:
(iii) catalytically hydrocracking higher boiling range paraffinic hydrocarbons produced in step (ii).

13. Hydrocarbons whenever provided by a process as claimed in claim 11 or claim 12.

14. A catalyst precursor whenever prepared by a process as defined in any one of claims 1 to 9.

15. A catalyst whenever prepared by a process as defined in claim 10.

16. Use of a catalyst as defined in claim 15 in a process for producing normally gaseous, normally liquid and optionally normally solid hydrocarbons from synthesis gas which comprises the steps of:
(i) providing the synthesis gas; and
(ii) catalytically converting the synthesis gas of step (i) at an elevated temperature and pressure to obtain the normally gaseous, normally liquid and optionally normally solid hydrocarbons.

17. A method of reducing the production of carbon dioxide as a by-product in a process for producing normally gaseous, normally liquid, and optionally normally solid hydrocarbons from synthesis gas as defined in claim 11 or claim 12, wherein the catalyst for step (ii) is formed from a catalyst precursor as defined in any one of the claim 1 to 9, or is a catalyst as defined in claim 10.
